(19) United States
(12) Patent Application Publication
(10) Pub. No.: US 2021/0198179 A1
(43) Pub. Date: Jul. 1, 2021

(54) COMPOUND AND COATING COMPOSITION EMPLOYING THE SAME

(71) Applicant: INDUSTRIAL TECHNOLOGY RESEARCH INSTITUTE, Hsinchu (TW)

(72) Inventors: Ya-I HSU, Taoyuan City (TW); Yuan-Chang HUANG, Hsinchu City (TW); Yi-Che SU, Zhubei City (TW); Wei-Cheng TANG, Hsinchu City (TW)

(73) Assignee: INDUSTRIAL TECHNOLOGY RESEARCH INSTITUTE, Hsinchu (TW)

(21) Appl. No.: 16/730,470

(22) Filed: Dec. 30, 2019

(51) Int. Cl. C07C 69/734; C07C 43/13; C09D 175/04; C09D 163/00; C09D 167/00; C09D 133/08

(52) U.S. Cl. CPC C07C 69/734; C07C 43/135; C09D 133/08; C09D 163/00; C09D 167/00; C09D 175/04

(57) ABSTRACT

A compound serving as coalescing agent and a coating composition employing the compound are provided. The compound has a structure represented by Formula (I)

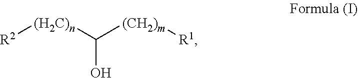

wherein n is 0, 1, 2, or 3; m is 0, 1, 2, or 3; R¹ is

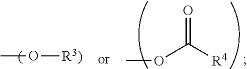

R² is

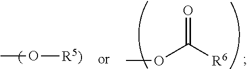

R³, R⁴, R⁵, and R⁶ are independently C₁₋₁₂ alkyl group; and, R¹ is distinct from R² when n is equal to m.

### TECHNICAL FIELD

The disclosure relates to a compound and a coating composition employing the same.

### BACKGROUND

In recent times, water-based coating compositions are widely applied in the construction industry for decorative and protective purposes.

Traditionally, coalescing agents are used in substantial volumes, particularly in latex coating compositions based on small particles of synthetic polymers such as polyacrylate. These coalescing agents are added to a coating in order to improve film formation. Their function derives from the plasticizing action which the coalescing agent has on the latex particles, enabling these particles to flow together and to form a continuous film. This film has optimum properties after the evaporation of the water. Significant in the context of the formation of a film is the temperature referred to as the film-forming temperature, at which (or below which) the polymer particles flow together to form a film. The coalescing agents lower the film-forming temperature of the polymer.

Conventional coalescing agents, such as 2,2,4-trimethyl-1,3-pentanediol monoisobutyrate or ethylene glycol monobutyl ether, have not met the latest international regulations on non-volatile organic compounds (which evaporate at 260° C. or higher as determined by the method provided in ASTM D6886). Since the volatile organic compound (VOC) leads to serious problems with environmental pollution, in the State of California, the use and content of VOCs in coating compositions are subject to regulation by the South Coast Air Quality Management District (SCAQMD). The SCAQMD has mandated on manufacturers of coating compositions to reduce the VOC content in coating compositions from 150 g/L to 50 g/L.

As a result of the increasingly stringent regulations on VOCs in coating compositions, manufacturers of coating compositions have embarked on a quest to develop low-VOC or VOC-free coating compositions while maintaining the physical properties that are obtained when a volatile coalescing agent is used. Therefore, there is a need to develop a non-volatile organic compound, which serves as the coalescing agent for a coating composition, in order to overcome the problems mentioned above.

### SUMMARY

A detailed description is given in the following embodiments.

According to embodiments of the disclosure, the disclosure provides a compound. The compound may have a structure represented by Formula (I)

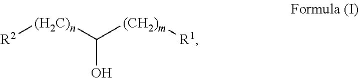

wherein n can be 0, 1, 2, or 3; m can be 0, 1, 2, or 3; R¹ may be

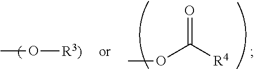

### R² may be

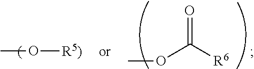

R³, R⁴, R⁵, and R⁶ can be independently C₁₋₁₂ alkyl group; and, R¹ is distinct from R² when n is equal to m.

According to embodiments of the disclosure, the disclosure also provides a coating composition. The coating composition can include an aqueous resin and a coalescing agent, wherein the coalescing agent may be a compound having a structure represented by Formula (I).

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

According to embodiments of the disclosure, the disclosure provides a compound, having a structure represented by Formula (I)

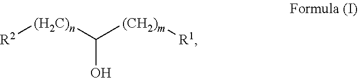

wherein, n can be 0, 1, 2, or 3; m can be 0, 1, 2, or 3; R¹ may be

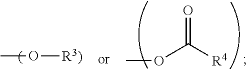

### R² may be

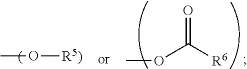

R³, R⁴, R⁵, and R⁶ can be independently C₁₋₁₂ alkyl group; and, R¹ is distinct from R² when n is equal to m. Since the compounds of Formula (I) of the disclosure are compounds having a secondary alcohol

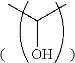

moiety and two functional groups (which are selected from a group of C₂₋₁₃ alkylcarbonyloxy and C₁₋₁₂ alkoxy), the boiling point of the compound of the disclosure can be modified to be equal to or greater than 260° C. Therefore, the compound of the disclosure can be a non-volatile organic compound and is suitable to serve as a coalescing agent. As a result, the VOC content of the coating composition would not be increased when adding the compound of the disclosure thereinto.

In addition, since the compound having a structure represented by Formula (I) of the disclosure has an asymmetric chemical structure, the melting point of the compound of the disclosure can be adjusted to be equal to or less than 30° C. As a result, the melting point requirement of the coalescing agent, which is suitable for using in a coating composition, can be met.

According to embodiments of the disclosure, due to the chemical structure represented by Formula (I), the compound of the disclosure exhibits good compatibility with the aqueous resin. Therefore, the coating composition employing the compound of the disclosure exhibits good film-forming ability, and the minimum film forming temperature of the coating composition can be reduced (resulting in reducing the operating temperature of the coating composition and reducing the amount of coalescing agent). It should be noted that, when the difference between the Hansen solubility parameters of the coalescing agent and the Hansen solubility parameters of the aqueous resin is relatively large, the compatibility of the coalescing agent with the aqueous resin is inferior. The poor compatibility results in low film-forming ability of the coating composition and insufficient hardness, elongation and tensile strength of the film prepared from the coating composition.

According to embodiments of the disclosure, in comparison with the compound of Formula (I), the compound, which has a structure similar to the structure represented by Formula (I) but does not have a secondary alcohol

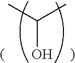

moiety, may have a relatively low boiling point and a relatively low hydrogen bonding parameter δₕ of the Hansen solubility parameters. Accordingly, said compound may exhibit a poor compatibility with the aqueous resin, thereby limiting the application thereof.

According to embodiments of the disclosure, in comparison with the compound of Formula (I), the secondary alcohol compound, which has one functional group or at least three functional groups (wherein the functional group is selected from a group of C₂₋₁₃ alkylcarbonyloxy and C₁₋₁₂ alkoxy), may have a polarity parameter δₚ (of the Hansen solubility parameters) which does not match the polarity parameter δₚ of the aqueous resin. As a result, said compound may exhibit a poor compatibility with the aqueous resin, thereby limiting the application thereof. Calculations for evaluating the various HSP (Hansen solubility parameters) values can be performed using a commercially available software package such as HSPiP (Hansen Solubility Parameters in Practice, available from the Hansen Solubility Parameters internet site, currently in the 4th edition).

According to embodiments of the disclosure, C₂₋₁₃ alkylcarbonyloxy may have a structure represented by

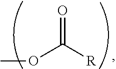

wherein R is C₁₋₁₂ alkyl group. According to embodiments of the disclosure, C₁₋₁₂ alkoxy has a structure represented by O—R), wherein R is C₁₋₁₂ alkyl group. According to embodiments of the disclosure, C₁₋₁₂ alkyl group can be a linear or branched alkyl group having 1-12 carbon atoms.

According to embodiments of the disclosure, C₁₋₁₂ alkyl group may be methyl, ethyl, propyl, butyl, pentyl, hexyl, heptyl, octyl, nonyl, decyl, undecyl, dodecyl, or an isomer thereof.

According to embodiments of the disclosure, in Formula (I), since the compound of the disclosure has an asymmetric chemical structure, R¹ is distinct from R² when n is equal to m.

According to embodiments of the disclosure, the compound of the disclosure may be

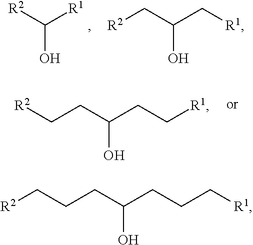

wherein R¹ may be

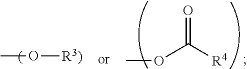

### R² may be

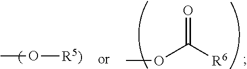

R³, R⁴, R⁵, and R⁶ can be independently C₁₋₁₂ alkyl group; and, R¹ is distinct from R².

According to embodiments of the disclosure, even though R¹ and R² are the same, the compound of the disclosure still has an asymmetric chemical structure when n is not equal to m. According to embodiments of the disclosure, the compound of the disclosure may be

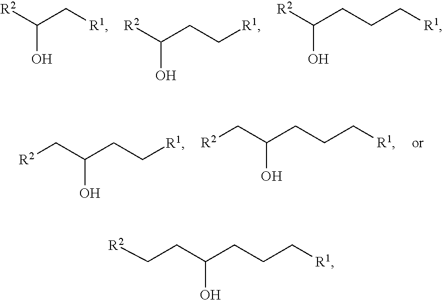

wherein R¹ may be

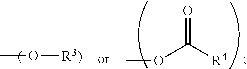

### R² may be

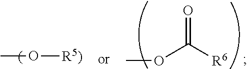

and, R³, R⁴, R⁵, and R⁶ can be independently C₁₋₁₂ alkyl group.

According to embodiments of the disclosure, the compound of the disclosure may be prepared from a compound with an asymmetric chemical structure. The compound of the disclosure may be

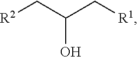

wherein R¹ may be

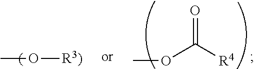

### R² may be

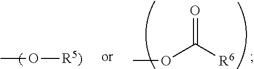

R³, R⁴, R⁵, and R⁶ can be independently C₁₋₁₂ alkyl group; and, R¹ is distinct from R².

According to embodiments of the disclosure, when each of R³, R⁴, R⁵, and R⁶ is an alkyl group having at least 13 carbon atoms, the compound may exhibit a relatively low polarity parameter (δ_{P}) of the Hansen solubility parameters. As a result, the solubility parameters of the compound are unable to match the solubility parameters of the aqueous resin.

According to embodiments of the disclosure, the compound of the disclosure may be

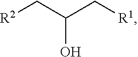

wherein R¹ may be

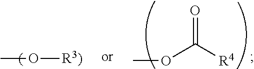

### R² may be

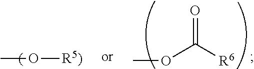

R³ and R⁴ can be independently C₁₋₆ alkyl group; R⁵ and R⁶ can be independently C₁₋₁₂ alkyl group; and, R¹ is distinct from R². Accordingly, the compound having the aforementioned structure can have solubility parameters which match the solubility parameters of the aqueous resin (such as an acrylic resin). As a result, the coating composition employing the compound of the disclosure exhibits good film-forming ability, and the film prepared from the coating composition also exhibits improved hardness, elongation and tensile strength. According to embodiments of the disclosure, C₁₋₆ alkyl group may be methyl, ethyl, propyl, butyl, pentyl, hexyl, or an isomer thereof.

According to embodiments of the disclosure, the compound may be

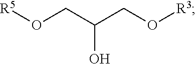

R³ and R⁵ can be independently C₁₋₁₂ alkyl group; and, R³ is distinct from R⁵.

According to embodiments of the disclosure, the compound may be

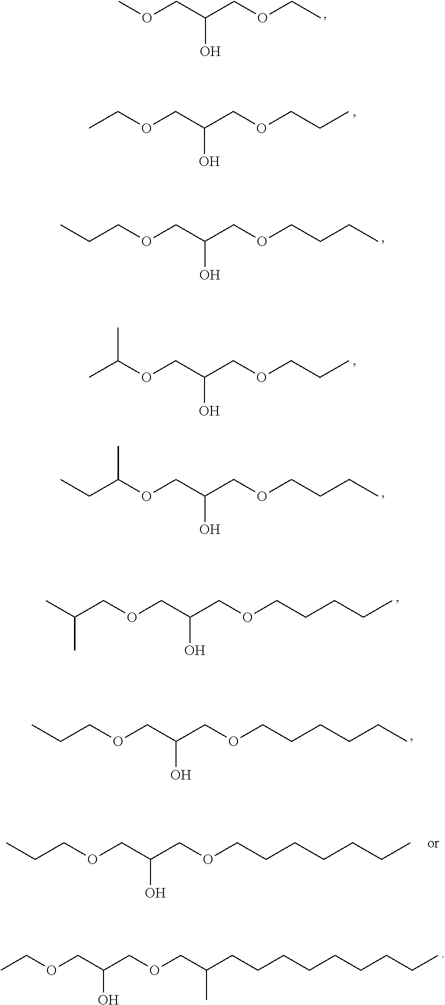

According to embodiments of the disclosure, the compound may be

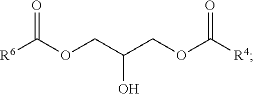

R⁴ and R⁶ can be independently C₁₋₁₂ alkyl group; and, R⁴ is distinct from R⁶.

According to embodiments of the disclosure, the compound may be

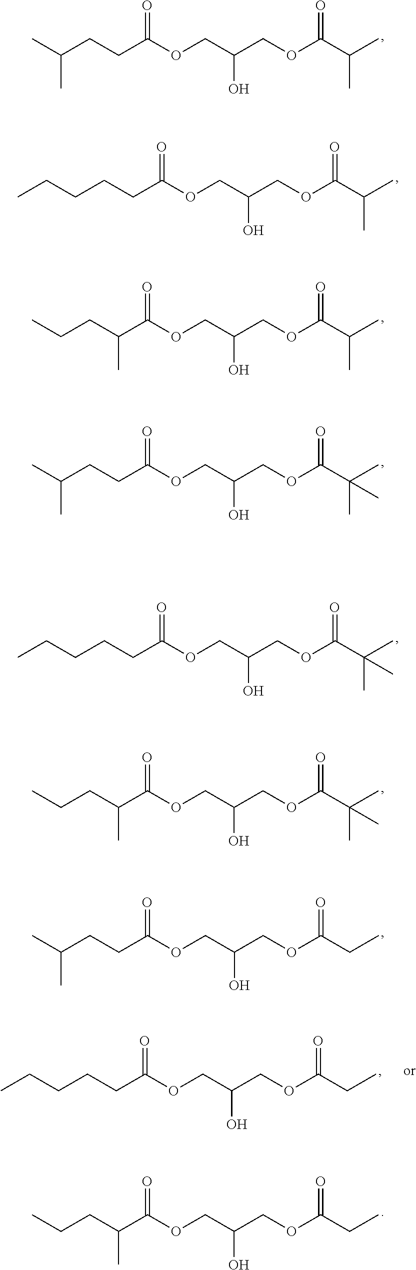

According to embodiments of the disclosure, the compound may be

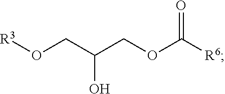

and, R³ and R⁶ can be independently C₁₋₁₂ alkyl group. When the compound of the disclosure is a secondary alcohol compound which has one ester moiety and one ether moiety, the solubility parameters of the compound of the disclosure are apt to match the solubility parameters of the aqueous resin (such as an acrylic resin), thereby improving the compatibility of the coalescing agent with the aqueous resin. Accordingly, the coating composition employing the compound of the disclosure exhibits good film-forming ability, and the film prepared from the coating composition also exhibits improved hardness, elongation, and tensile strength.

According to embodiments of the disclosure, the compound may be

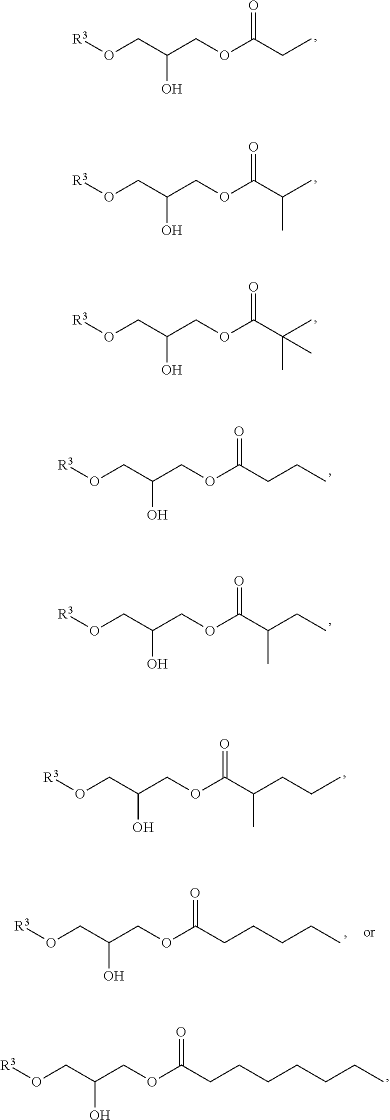

and R³ may be C₁₋₁₂ alkyl group.

According to embodiments of the disclosure, the compound may be

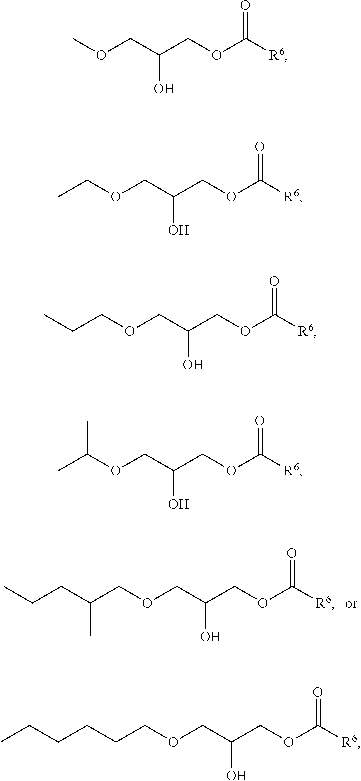

and R⁶ may be C₁₋₁₂ alkyl group.

According to embodiments of the disclosure, the compound may be

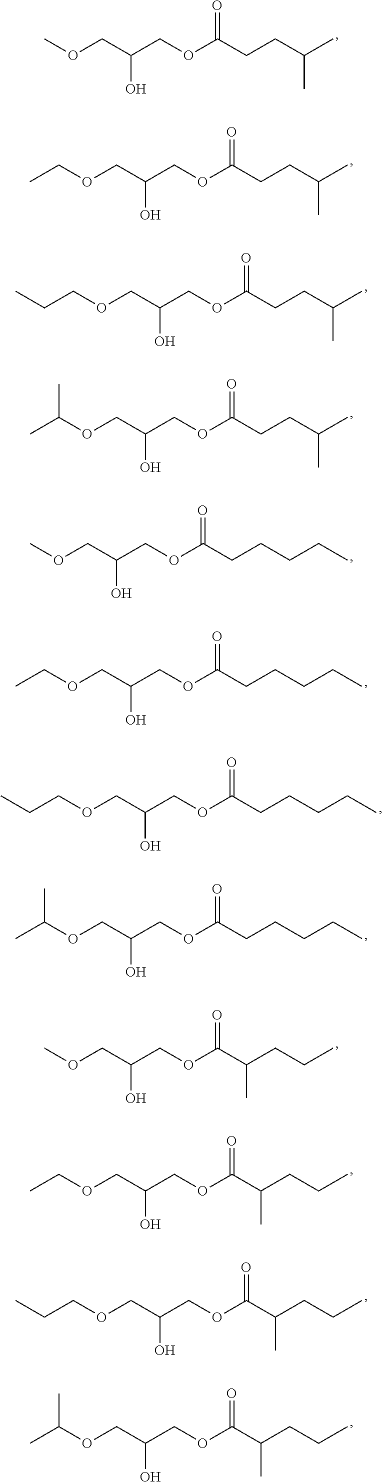

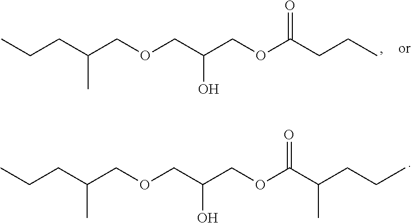

According to embodiments of the disclosure, the compound may be

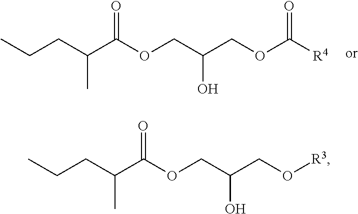

wherein R³ and R⁴ may be C₁₋₁₂ alkyl group.

According to embodiments of the disclosure, the disclosure also provides a coating composition. The coating composition can include an aqueous resin and a coalescing agent, wherein the coalescing agent may be a compound having a structure represented by Formula (I).

According to embodiments of the disclosure, the aqueous resin is epoxy resin, polyurethane resin, acrylic resin, polyester resin, or a combination thereof. According to embodiments of the disclosure, the number average molecular weight of the aqueous resin of the disclosure may be, but is not limited to, from 500 to 1,000,000.

According to embodiments of the disclosure, since the compound of the disclosure exhibits good compatibility with the aqueous resin, the amount of coalescing agent may be reduced in a coating composition when the compound of the disclosure serves as a coalescing agent. According to embodiments of the disclosure, the weight ratio of the coalescing agent to the aqueous resin may be from 0.1:100 to 10:100, such as 0.1:100, 0.2:100, 0.5:100, 0.8:100, 1:100, 2:100, 3:100, 5:100, 8:100, or 10:100.

According to embodiments of the disclosure, the coating composition of the disclosure can further include an additive, wherein the weight percentage of the additive may be from 0.01 wt % to 40 wt %, based on the weight of the aqueous resin. According to embodiments of the disclosure, the additive may be, for example, dye, pigment, antioxidant, stabilizer, fixing agent, dispersant, or a combination thereof.

According to embodiments of the disclosure, since the compound of the disclosure is a non-volatile organic compound, the VOC content of the coating composition would not be increased when the compound of the disclosure serves as a coalescing agent and is added into the coating composition. As a result, the object for preparing a low VOC coating composition or zero VOC coating composition is achieved. According to embodiments of the disclosure, since the coating composition of the disclosure exhibits a good film-forming ability, the film prepared from the coating composition of the disclosure also exhibits superior hardness, elongation and tensile strength.

Below, exemplary embodiments will be described in detail with reference to the accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

### EXAMPLES

Solubility Parameters Evaluation of Compounds

By means of computer software HSPiP (Hansen Solubility Parameters in Practice, version 4.1), the Hansen solubility parameters, boiling point (BP) and melting point (MP) of various secondary alcohol compounds (which have one ester moiety and one ether moiety) were evaluated, and the results are shown in Table 1.

TABLE 1

| | | | | | BP | MP |
|---|---|---|---|---|---|---|
| compound | δ_{d} | δₚ | δₕ | δₜ | (° C.) | (° C.) |
| 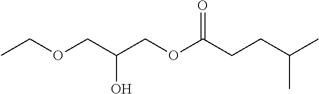 | 16.1 | 5.7 | 8.2 | 18.9 | 262.8 | 15.6 |
| 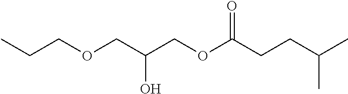 [SMILES: CCCOCC(O)COC(=O)CCC(C)C] | 16.1 | 5.2 | 7.6 | 18.5 | 274.7 | 17.5 |
| 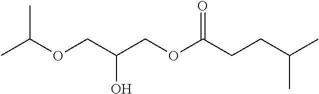 [SMILES: CC(C)CCC(=O)OCC(O)COC(C)C] | 16.0 | 5.1 | 7.2 | 18.2 | 271.3 | 25.4 |
| 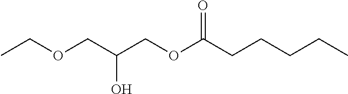 | 16.3 | 5.9 | 9.0 | 19.5 | 269.4 | 22.5 |
| 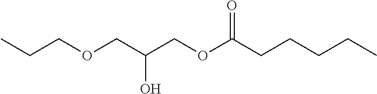 [SMILES: CCCCCC(=O)OCC(O)COCCC] | 16.3 | 5.4 | 8.4 | 19.1 | 281.2 | 22.2 |
| 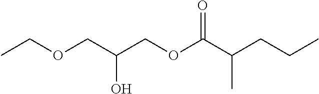 | 16.2 | 5.4 | 8.2 | 19.0 | 260.6* | 2.7 |
| 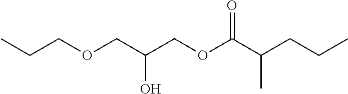 [SMILES: CCCOCC(O)COC(=O)C(C)CCC] | 16.2 | 5.0 | 7.6 | 18.6 | 264.2* | 4.1 |
| 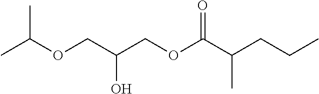 [SMILES: CCCC(C)C(=O)OCC(O)COC(C)C] | 16.1 | 4.9 | 7.2 | 18.3 | 260.8 | 11.9 |
| 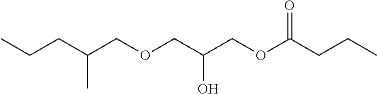 [SMILES: CCCC(=O)OCC(O)COCC(C)CCC] | 16.2 | 4.8 | 7.3 | 18.4 | 291.1 | 3.5 |
| 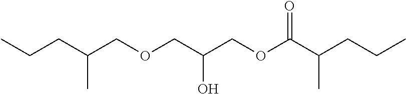 [SMILES: CCCC(C)COCC(O)COC(=O)C(C)CCC] | 16.2 | 4.1 | 6.5 | 17.9 | 314.1 | 1.4 |
| 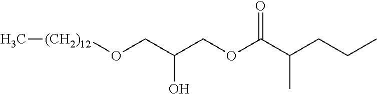 | 16.1 | 3.5 | 4.9 | 17.2 | 413.2 | 21.3 |

*the boiling point (i.e. initial boiling point) of the compound was determined by ASTM D-86.

As shown in Table 1, when the secondary alcohol compounds, which have one ester moiety and one ether moiety, have a structure represented by Formula (I), said compounds have a boiling point greater than 260° C., and a melting point less than or equal to 30° C. In addition, the secondary alcohol compounds, which have one ester moiety and one ether moiety and have a structure represented by Formula (I), may have a solubility parameter δₜ from 17.9 to 19.5, and a polarity parameter δₚ from 4.1 to 6.

By means of computer software HSPiP (Hansen Solubility Parameters in Practice, version 4.1), the Hansen solubility parameters, boiling point (BP) and melting point (MP) of various secondary alcohol compounds (which have two ester moieties) were evaluated, and the results are shown in Table 2.

TABLE 2

| | | | | | BP | MP |
|---|---|---|---|---|---|---|
| compound | δ_{d} | δₚ | δₕ | δₜ | (° C.) | (° C.) |
| 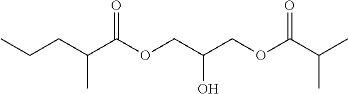 [SMILES: CCCC(C)C(=O)OCC(O)COC(=O)C(C)C] | 16.3 | 4.8 | 7.6 | 18.6 | 299.7* | 15 |
| 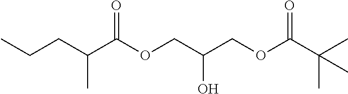 [SMILES: CCCC(C)C(=O)OCC(O)COC(=O)C(C)(C)C] | 16.0 | 4.2 | 3.3 | 16.9 | 316 | 27 |
| 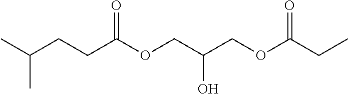 [SMILES: CCC(=O)OCC(O)COC(=O)CCC(C)C] | 16.2 | 5.6 | 8.6 | 19.2 | 299 | 21 |
| 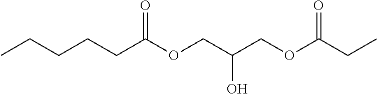 [SMILES: CCCCCC(=O)OCC(O)COC(=O)CC] | 16.5 | 5.8 | 9.4 | 19.8 | 306 | 26 |
| 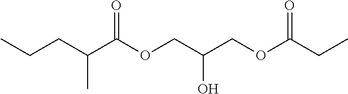 [SMILES: CCCC(C)C(=O)OCC(O)COC(=O)CC] | 16.4 | 5.4 | 8.6 | 19.3 | 293.6* | 9 |

*the boiling point (i.e. initial boiling point) of the compound was determined by ASTM D-86.

As shown in Table 2, when the secondary alcohol compounds, which have two ester moieties, have a structure represented by Formula (I), said compounds have a boiling point greater than 260° C., and a melting point less than or equal to 30° C. In addition, the secondary alcohol compounds, which have two ester moieties and have a structure represented by Formula (I), may have a solubility parameter δₜ from 16.9 to 19.8, and a polarity parameter δₚ from 4.2 to 5.8.

By means of computer software HSPiP (Hansen Solubility Parameters in Practice, version 4.1), the Hansen solubility parameters, boiling point (BP) and melting point (MP) of various secondary alcohol compounds (which have two ether moieties) were evaluated, and the results are shown in Table 3.

TABLE 3

| | | | | | BP | MP |
|---|---|---|---|---|---|---|
| compound | δ_{d} | δₚ | δₕ | δₜ | (° C.) | (° C.) |
| 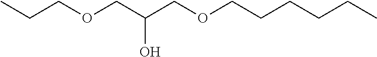 | 16.0 | 5.3 | 7.4 | 18.4 | 260.8 | −2.4 |
| 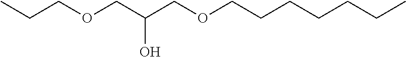 [SMILES: CCCCCCCOCC(O)COCCC] | 16.0 | 5.2 | 7.0 | 18.2 | 274.1 | 12.3 |

As shown in Table 3, when the secondary alcohol compounds, which have two ether moieties, have a structure represented by Formula (I), said compounds have a boiling point greater than 260° C., and a melting point less than or equal to 30° C. In addition, the secondary alcohol compounds, which have two ether moieties and have a structure represented by Formula (I), may have a solubility parameter δₜ from 18.0 to 19.4, and a polarity parameter δₚ from 5.0 to 6.4.

Preparation of Compound

### Example 1

9.25 g of N,N′-dicyclohexylcarbodiimide (DCC), 0.28 g of 4-dimethylaminopyridine (DMAP), and 40 g of tetrahydrofuran (THF) were added into a reaction bottle. After stirring at room temperature, 3.55 g of glycerol was added into the reaction bottle. Next, 3 g of propionic acid was slowly added into the reaction bottle. After stirring for 3 hours at room temperature, 4.7 g of 2-methylpentanoic acid was added into the reaction bottle. After the reaction was complete, the result was purified by column chromatography. Compound (1) (having a structure represented by

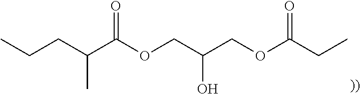 [SMILES: CCCC(C)C(=O)OCC(O)COC(=O)CC]

was obtained.

The result of nuclear magnetic resonance spectrometry of Compound (1) is shown below. ¹H NMR (CDCl₃, 400 MHz): δ 0.88 (3H, t, J=6.5 Hz), 1.06 (3H, t, J=7.2 Hz), 1.13 (3H, d, J=7.0 Hz), 1.23-1.35 (2H, 1.29 (tq, J=7.3, 6.5 Hz), 1.29 (tq, J=7.3, 6.5 Hz)), 1.44-1.56 (2H, 1.50 (q, J=7.3 Hz), 1.50 (q, J=7.3 Hz)), 2.26-2.30 (2H, 2.28 (q, J=7.2 Hz), 2.28 (q, J=7.2 Hz)), 2.40 (1H, tq, J=7.3, 7.0 Hz), 4.09 (1H, quint, J=6.5 Hz), 4.51-4.57 (4H, 4.55 (d, J=6.5 Hz), 4.53 (d, J=6.5 Hz), 4.53 (d, J=6.5 Hz), 4.55 (d, J=6.5 Hz)).

### Example 2

9.25 g of N,N′-dicyclohexylcarbodiimide (DCC), 0.28 g of 4-dimethylaminopyridine (DMAP), and 40 g of tetrahydrofuran (THF) were added into a reaction bottle. After stirring at room temperature, 3.55 g of glycerol was added into the reaction bottle. Next, 3.57 g of isobutyric acid was slowly added into the reaction bottle. After stirring for 3 hours at room temperature, 4.7 g of 2-methylpentanoic acid was added into the reaction bottle. After the reaction was complete, the result was purified by column chromatography. Compound (2) (having a structure represented by

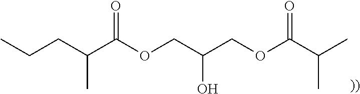 [SMILES: CCCC(C)C(=O)OCC(O)COC(=O)C(C)C]

was obtained.

The result of nuclear magnetic resonance spectrometry of Compound (2) is shown below. NMR (CDCl₃, 400 MHz): δ 0.88 (3H, t, J=6.5 Hz), 1.09-1.15 (9H, 1.11 (d, J=7.0 Hz), 1.13 (d, J=7.0 Hz), 1.11 (d, J=7.0 Hz)), 1.23-1.35 (2H, 1.29 (tq, J=7.4, 6.5 Hz), 1.29 (tq, J=7.4, 6.5 Hz)), 1.44-1.56 (2H, 1.50 (td, J=7.4, 7.3 Hz), 1.50 (td, J=7.4, 7.3 Hz)), 2.34-2.46 (2H, 2.41 (sept, J=7.0 Hz), 2.40 (tq, J=7.3, 7.0 Hz)), 4.09 (1H, quint, J=6.5 Hz), 4.51-4.56 (4H, 4.53 (d, J=6.5 Hz), 4.54 (d, J=6.5 Hz), 4.54 (d, J=6.5 Hz), 4.53 (d, J=6.5 Hz)).

### Example 3

9.25 g of N,N′-dicyclohexylcarbodiimide (DCC), 0.28 g of 4-dimethylaminopyridine (DMAP), and 40 g of tetrahydrofuran (THF) were added into a reaction bottle. After stirring at room temperature, 3.55 g of glycerol was added into the reaction bottle. Next, 4.4 g of bromoethane was slowly added into the reaction bottle. After stirring for 3 hours at room temperature, 4.7 g of 2-methylpentanoic acid was added into the reaction bottle. After the reaction was complete, the result was purified by column chromatography. Compound (3) (having a structure represented by

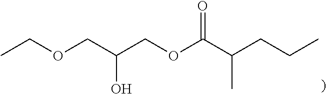

was obtained.

The result of nuclear magnetic resonance spectrometry of Compound (3) is shown below. NMR (CDCl₃, 400 MHz): δ 0.88 (3H, t, J=6.5 Hz), 1.13 (3H, d, J=7.0 Hz), 1.20-1.35 (4H, 1.24 (t, J=7.0 Hz), 1.29 (tq, J=7.4, 6.5 Hz)), 1.29 (1H, tq, J=7.4, 6.5 Hz), 1.44-1.56 (2H, 1.50 (td, J=7.4, 7.3 Hz), 1.50 (td, J=7.4, 7.3 Hz)), 2.40 (1H, tq, J=7.3, 7.0 Hz), 3.39-3.44 (2H, 3.41 (q, J=7.0 Hz), 3.41 (q, J=7.0 Hz)), 3.46-3.49 (2H, 3.47 (d, J=5.4 Hz), 3.47 (d, J=5.4 Hz)), 4.05 (1H, tt, J=6.5, 5.4 Hz), 4.51-4.56 (2H, 4.54 (d, J=6.5 Hz), 4.54 (d, J=6.5 Hz)).

### Example 4

30 g of glycerol and 14.7 g of tetrabutyl ammonium bromide were added into a reaction bottle, and then dissolved in 600 mL of potassium hydroxide aqueous solution (with a concentration of 33%). After stirring, 4.98 g of bromopropane was added into the reaction bottle. Next, after stirring at 110° C. for 24 hours, 150 mL of 1-hexanol was added into the reaction bottle. After stirring at 110° C. for 12 hours, the result was purified by column chromatography. Compound (4) (having a structure represented by

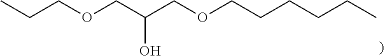

was obtained.

The result of nuclear magnetic resonance spectrometry of Compound (4) is shown below. NMR (CDCl₃, 400 MHz): δ 0.82-0.97 (6H, 0.93 (t, J=7.6 Hz), 0.86 (t, J=7.0 Hz)), 1.19-1.42 (5H, 1.36 (tt, J=7.0, 5.7 Hz), 1.28 (h, J=7.0 Hz), 1.26 (quint, J=7.0 Hz), 1.26 (quint, J=7.0 Hz), 1.36 (tt, J=7.0, 5.7 Hz)), 1.28 (1H, h, J=7.0 Hz), 1.58-1.79 (4H, 1.72 (tt, J=7.2, 5.7 Hz), 1.64 (qt, J=7.6, 7.2 Hz), 1.64 (qt, J=7.6, 7.2 Hz), 1.72 (tt, J=7.2, 5.7 Hz)), 3.31-3.41 (4H, 3.37 (t, J=7.2 Hz), 3.35 (t, J=7.2 Hz), 3.35 (t, J=12 Hz), 3.37 (t, J=7.2 Hz)), 3.44-3.48 (4H, 3.46 (d, J=5.5 Hz), 3.46 (d, J=5.5 Hz), 3.46 (d, J=5.5 Hz), 3.46 (d, J=5.5 Hz)), 3.95 (1H, quint, J=5.5 Hz).

### Example 5

30 g of glycerol and 14.7 g of tetrabutyl ammonium bromide were added into a reaction bottle, and then dissolved in 600 mL of potassium hydroxide aqueous solution (with a concentration of 33%). After stirring, 4.98 g of bromopropane was added into the reaction bottle. Next, after stirring at 110° C. for 24 hours, 150 mL of 1-heptanol was added into the reaction bottle. After stirring at 110° C. for 12 hours, the result was purified by column chromatography. Compound (5) (having a structure represented by

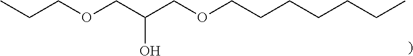 [SMILES: CCCCCCCOCC(O)COCCC]

was obtained.

The result of nuclear magnetic resonance spectrometry of Compound (5) is shown below. ¹H NMR (CDCl₃, 400 MHz): δ 0.82-0.97 (6H, 0.86 (t, J=7.0 Hz), 0.93 (t, J=7.6 Hz)), 1.16-1.43 (7H, 1.27 (quint, J=7.0 Hz), 1.36 (tt, J=7.0, 5.7 Hz), 1.28 (h, J=7.0 Hz), 1.28 (h, J=7.0 Hz), 1.24 (quint, J=7.0 Hz), 1.24 (quint, J=7.0 Hz), 1.27 (quint, J=7.0 Hz)), 1.36 (1H, tt, J=7.0, 5.7 Hz), 1.58-1.80 (4H, 1.73 (tt, J=7.2, 5.7 Hz), 1.64 (qt, J=7.6, 7.2 Hz), 1.64 (qt, J=7.6, 7.2 Hz), 1.73 (tt, J=7.2, 5.7 Hz)), 3.31-3.39 (4H, 3.35 (t, J=7.2 Hz), 3.35 (t, J=7.2 Hz), 3.35 (t, J=7.2 Hz), 3.35 (t, J=7.2 Hz)), 3.44-3.48 (4H, 3.46 (d, J=5.5 Hz), 3.46 (d, J=5.5 Hz), 3.46 (d, J=5.5 Hz), 3.46 (d, J=5.5 Hz)), 3.96 (1H, quint, J=5.5 Hz).

Coating Composition

### Example 6

100 parts by weight of aqueous acrylic resin (sold by Eternal Materials Co., Ltd. with a trade number of ETERSOL 1119) (having a minimum film forming temperature (MFFT) of about 34° C.) and 2 parts by weight of Compound (1) (serving as a coalescing agent) were homogeneously mixed by a mixer, obtaining a mixture. Next, the mixture was defoamed by a centrifuge (at 2,000 rpm for 2 minutes), obtaining Coating Composition (1). The minimum film forming temperature (MFFT) of Coating Composition (1) was measured, and the temperature difference (ΔT_{MFFT}) between the MFFT of the aqueous acrylic resin and the MFFT of the Coating Composition (1) was calculated. The result is shown in Table 4. The temperature difference (ΔT_{MFFT}) between the MFFT of the aqueous acrylic resin and the MFFT of the Coating Composition (1) was determined by the following equation: ΔT_{MFFT}=T_{R}−T_{C}, wherein T_{R} is the minimum film forming temperature (MFFT) of the aqueous acrylic resin, and T_{C} is the minimum film forming temperature (MFFT) of Coating Composition (1). The minimum film forming temperature (MFFT) is determined according to ASTM D2354.

Next, Coating Composition (1) was coated on a glass substrate and dried at room temperature for 120 minutes, obtaining a film (with a thickness of 20˜30 μm). The pendulum hardness, tensile strength and elongation of the film were evaluated and the results are shown in Table 4. The pendulum hardness is determined according to ASTM D 4366. The tensile strength and elongation are determined by universal tensile machine according to ASTM D412 and ASTM D624.

### Example 7

Example 7 was performed in the same manner as Example 6, except that Compound (1) was replaced with Compound (2), obtaining Coating Composition (2). The minimum film forming temperature (MFFT) of Coating Composition (2) was measured, and the temperature difference (ΔT_{MFFT}) between the MFFT of the aqueous acrylic resin and the MFFT of the Coating Composition (2) was calculated. The result is shown in Table 4.

Next, Coating Composition (2) was coated on a glass substrate and dried at room temperature for 120 minutes, obtaining a film (with a thickness of 20˜30 μm). The pendulum hardness, tensile strength and elongation of the film were evaluated and the results are shown in Table 4.

### Example 8

Example 8 was performed in the same manner as Example 6, except that Compound (1) was replaced with Compound (3), obtaining Coating Composition (3). The minimum film forming temperature (MFFT) of Coating Composition (3) was measured, and the temperature difference (ΔT_{MFFT}) between the MFFT of the aqueous acrylic resin and the MFFT of the Coating Composition (3) was calculated. The result is shown in Table 4.

Next, Coating Composition (3) was coated on a glass substrate and dried at room temperature for 120 minutes, obtaining a film (with a thickness of 20˜30 μm). The pendulum hardness, tensile strength and elongation of the film were evaluated and the results are shown in Table 4.

### Example 9

Example 9 was performed in the same manner as Example 6, except that Compound (1) was replaced with Compound (4), obtaining Coating Composition (4). The minimum film forming temperature (MFFT) of Coating Composition (4) was measured, and the temperature difference (ΔT_{MFFT}) between the MFFT of the aqueous acrylic resin and the MFFT of the Coating Composition (4) was calculated. The result is shown in Table 4.

Next, Coating Composition (4) was coated on a glass substrate and dried at room temperature for 120 minutes, obtaining a film (with a thickness of 20˜30 μm). The pendulum hardness, tensile strength and elongation of the film were evaluated and the results are shown in Table 4.

### Example 10

Example 10 was performed in the same manner as Example 6, except that Compound (1) was replaced with Compound (5), obtaining Coating Composition (5). The minimum film forming temperature (MFFT) of Coating Composition (5) was measured, and the temperature difference (ΔT_{MFFT}) between the MFFT of the aqueous acrylic resin and the MFFT of the Coating Composition (5) was calculated. The result is shown in Table 4.

Next, Coating Composition (5) was coated on a glass substrate and dried at room temperature for 120 minutes, obtaining a film (with a thickness of 20˜30 μm). The pendulum hardness, tensile strength and elongation of the film were evaluated and the results are shown in Table 4.

### Comparative Example 1

100 parts by weight of aqueous acrylic resin (sold by Eternal Materials Co., Ltd. with a trade number of ETERSOL 1119) (having a minimum film forming temperature (MFFT) of about 34° C.) was provided. The aqueous acrylic resin was defoamed by a centrifuge (at 2,000 rpm for 10 minutes), obtaining Coating Composition (6).

Next, Coating Composition (6) was coated on a glass substrate and dried at room temperature for 120 minutes, and there was no continuous film obtained.

TABLE 4

| | | ΔT | pendulum | tensile | |
|---|---|---|---|---|---|
| | | MFFT | hardness | strength | elongation |
| | coalescing agent | (° C.) | (sec) | (kgf/cm²) | (%) |
| Example 6 | 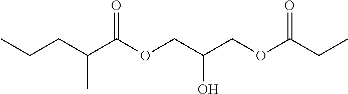 [SMILES: CCCC(C)C(=O)OCC(O)COC(=O)CC] | 7.3 | — | — | — |
| Example 7 | 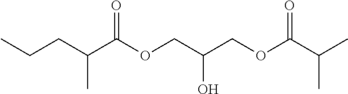 [SMILES: CCCC(C)C(=O)OCC(O)COC(=O)C(C)C] | 10.9 | 33.3 | — | — |
| Example 8 | 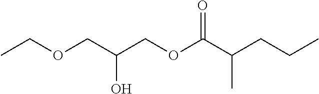 | 16.8 | 40.4 | 77.4 | 160.0 |
| Example 9 | 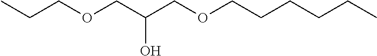 | 12.8 | 34.8 | — | — |
| Example 10 | 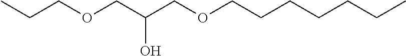 [SMILES: CCCCCCCOCC(O)COCCC] | 14.3 | 39.0 | — | — |

As shown in Table 4, when the coating composition includes the compound of the disclosure (serving as coalescing agent), the minimum film forming temperature of the coating composition can be reduced. In addition, the film prepared from the coating composition exhibits superior hardness, elongation and tensile strength.

It will be clear that various modifications and variations can be made to the disclosed methods and materials. It is intended that the specification and examples be considered as exemplary only, with the true scope of the disclosure being indicated by the following claims and their equivalents.

What is claimed is:

1. A compound, which has a structure represented by Formula (I) 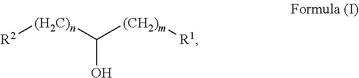
wherein n is 0, 1, 2, or 3; m is 0, 1, 2, or 3; R¹ is or 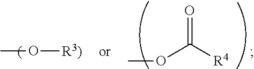
R² is 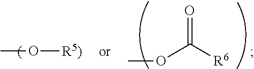
R³, R⁴, R⁵, and R⁶ are independently C1-12 alkyl group; and, R¹ is distinct from R² when n is equal to m.

2. The compound as claimed in claim 1, wherein the compound is 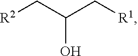
wherein R¹ is 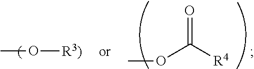
R² is 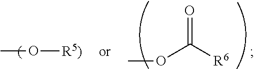
R³, R⁴, R⁵, and R⁶ are independently C₁₋₁₂ alkyl group; and, R¹ is distinct from R².

3. The compound as claimed in claim 2, wherein the compound is 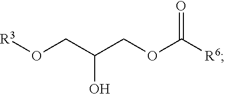
R³ and R⁶ are independently C₁₋₁₂ alkyl group.

4. The compound as claimed in claim 3, wherein the compound is 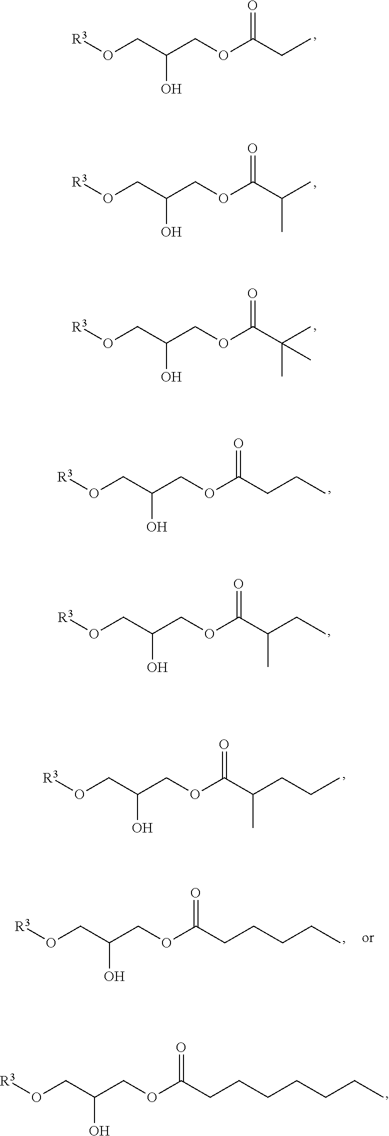
and R³ is C₁₋₁₂ alkyl group.

5. The compound as claimed in claim 3, wherein the compound is 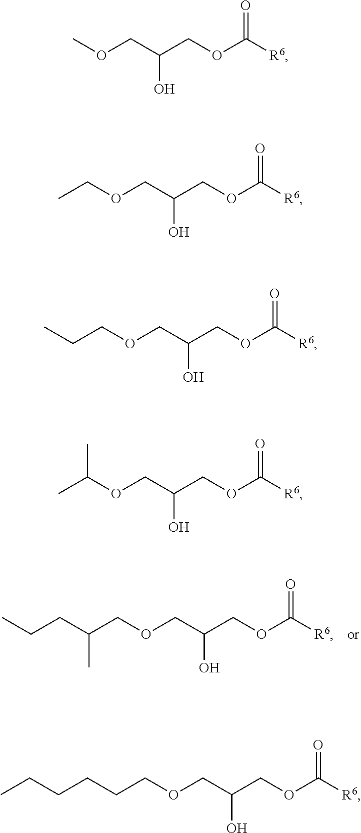
and R⁶ is C₁₋₁₂ alkyl group.

6. The compound as claimed in claim 3, wherein the compound is

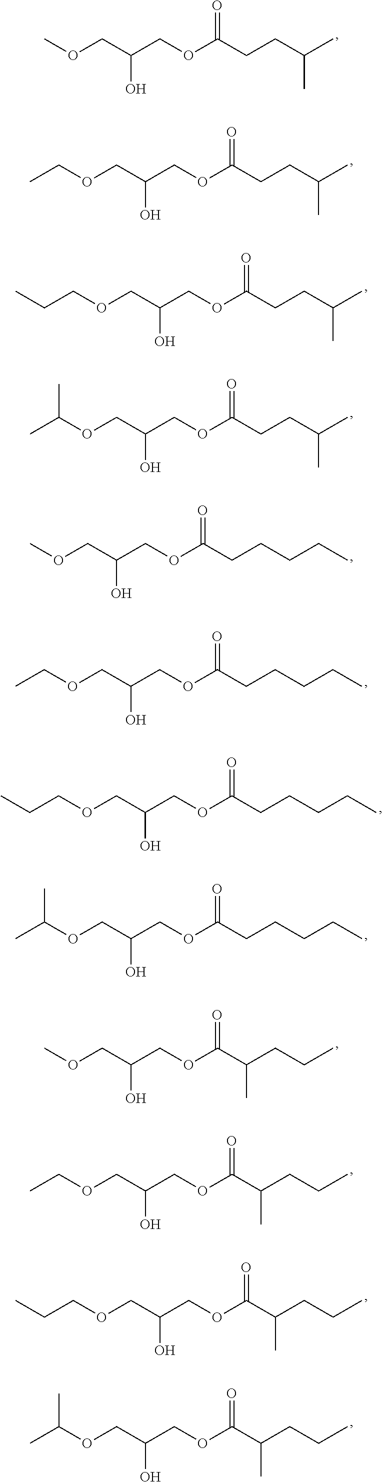

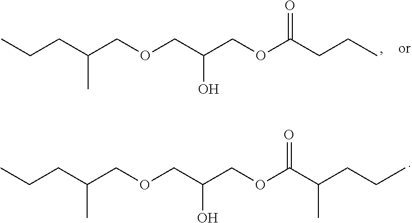

7. The compound as claimed in claim 2, wherein the compound is 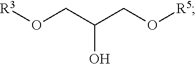
R³ and R⁵ are independently C₁₋₁₂ alkyl group; and, R³ is distinct from R⁵.

8. The compound as claimed in claim 7, wherein the compound is

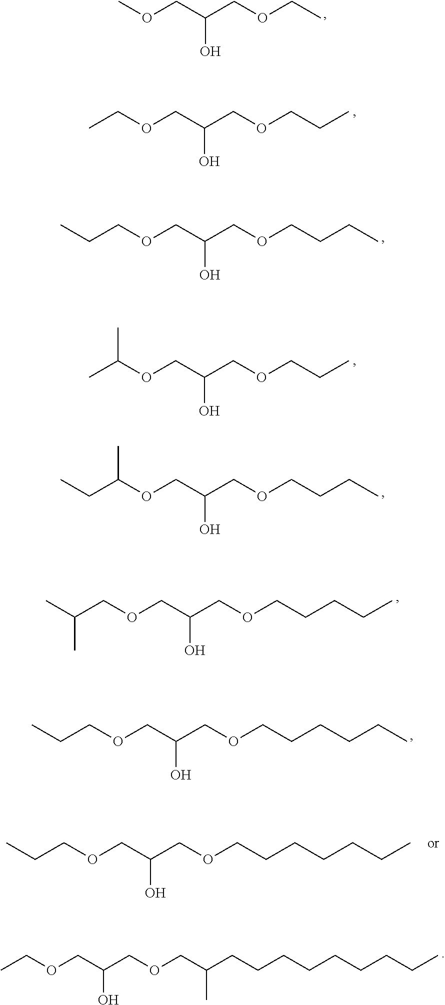

9. The compound as claimed in claim 2, wherein the compound is 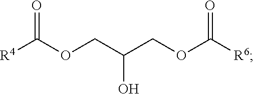
R⁴ and R⁶ are independently C₁₋₁₂ alkyl group; and, R⁴ is distinct from R⁶.

10. The compound as claimed in claim 9, wherein the compound is

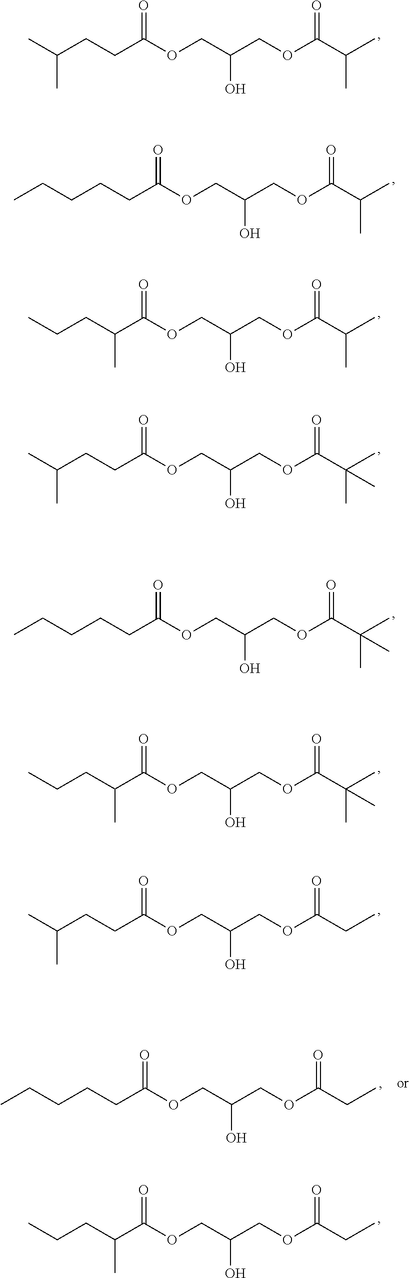

11. A coating composition, comprising:
an aqueous resin; and
a coalescing agent, wherein the coalescing agent is the compound as claimed in claim 1.

12. The coating composition as claimed in claim 11, wherein the aqueous resin is epoxy resin, polyurethane resin, acrylic resin, polyester resin, or a combination thereof.

13. The coating composition as claimed in claim 11, wherein the weight ratio of the coalescing agent to the aqueous resin is from 0.1:100 to 10:100.
